(19) United States
(12) Patent Application Publication
(10) Pub. No.: US 2026/0041824 A1
(43) Pub. Date: Feb. 12, 2026

(54) BREAST PUMP

(71) Applicant: X6 INNOVATIONS, Paris (FR)

(72) Inventors: QUENTIN SION, PARIS (FR); LOUISE AUMONT, PARIS (FR); ERICA PERRIER, PARIS (FR); DAVID THIBAULT, LEVALLOIS-PERRET (FR); MARINE KARAM, PARIS (FR)

(21) Appl. No.: 19/100,320

(22) PCT Filed: Aug. 3, 2023
(86) PCT No.: PCT/FR2023/051235

§ 371 Date: Jan. 31, 2025

(30) Foreign Application Priority Data
Aug. 3, 2022 (FR) FR2208039

(51) Int. Cl. A61M 1/06; A61M 1/00

(52) U.S. Cl. CPC A61M 1/067; A61M 1/066; A61M 1/81; A61M 2205/3389

(57) ABSTRACT

A breast pump (1) comprising: a casing (2); a milk collection unit (4) comprising a breast shield (6) and a tank (8) suitable for being fluidly connected, the milk collection unit (4) being suitable for being attached as a whole to the casing (2), after the breast shield (6) and the tank (8) have been fluidly connected, and being separated from the casing (2) while holding the breast shield (6) and the tank (8) fluidly connected; and a pump (18) arranged in the casing (8), the pump being configured to cause extraction of milk from a breast positioned against the breast shield when the milk collection unit (4) is attached to the casing (2), so as to cause a flow of milk from the breast shield (6) to the tank (8).

### FIELD OF THE INVENTION

The present invention relates to a breast pump.

### PRIOR ART

Document US20180361040 describes a breast pump comprising three parts: a casing containing a pump, a milk tank and a breast shield.

A bent passage is formed in the casing. The casing has two distinct accesses to the bent passage: a lower access and a frontal access.

To assemble the aforementioned three parts, it is necessary on the one hand to insert one end of the tank into the passage via the lower access, and on the other hand to insert one end of the breast shield into the passage via the frontal access, so that these two ends join in the passage and the breast shield and the tank are thus fluidly connected.

Subsequent starting of the pump when the breast shield is positioned against the breast of a user causes an extraction of milk from the breast to the breast shield, and the fluid connection between the breast shield and the tank allows the extracted milk to flow to the tank.

For hygienic reasons, it is necessary to regularly clean the different parts of the breast pump, in particular the tank and the breast shield, because these parts are contacted by the extracted milk.

Complete cleaning of the breast pump involves breaking the fluid connection between the breast shield and the tank at the bend of the cavity of the casing, before the breast shield and the tank are completely separated from the casing.

However, during this separation, drops of milk can come into contact with the casing, in particular at the bend of the cavity. This is not hygienic, and therefore requires cleaning of the casing, which is tedious.

### DISCLOSURE OF THE INVENTION

One object of the invention is to propose a breast pump which is easier to clean.

This object is attained by a breast pump comprising a casing, a milk collection unit, and a pump arranged in the casing. The milk collection unit comprises a breast shield and a tank suitable for being fluidly connected. Moreover, the milk collection unit is suitable for being attached as a whole to the casing, after the breast shield and the tank have been fluidly connected, and for being separated from the casing while still holding the breast shield and the tank fluidly connected. The pump is configured to cause extraction of milk from a breast when the milk collection unit is attached to the casing, so as to cause a flow of milk from the breast shield to the tank.

The breast pump can also comprise the following optional features, taken alone or combined together whenever this is technically possible.

Preferably, the breast pump comprises at least one ball spring plunger to attach the milk collection unit to the casing.

Preferably, the milk collection unit comprises a connector suitable for being removably attached to the tank and to the breast shield, the connector comprising a milk flow channel fluidly connecting the breast shield to the tank, when the tank and the breast shield are attached to the connector.

Preferably, the milk collection unit is suitable for being attached as a whole to the casing solely by the connector.

Preferably, the breast shield comprises a tube inserted into the connector when the breast shield is attached to the connector, the tube comprising an annular wall extending around an axis, and an aperture provided in the annular wall and leading into the milk flow channel when the tube is inserted into the connector.

Preferably, the milk collection unit also comprises a diaphragm configured to be deformed by the pump when the milk collection unit is attached to the casing, the deformation of the diaphragm causing reduced air pressure in the air channel when a breast is positioned against the breast shield.

Preferably, the diaphragm comprises an annular groove, and the connector comprises an annular rib able to be engaged in the annular groove, so as to attach the diaphragm to the connector.

Preferably, the connector comprises an air channel fluidly connecting the breast shield to the diaphragm, when the breast shield is attached to the connector.

Preferably, the breast shield comprises a tube inserted into the connector when the breast shield is attached to the connector, the tube comprising an annular wall extending around an axis and an aperture provided in the annular wall and leading into the air channel when the tube is inserted into the connector.

Preferably, the aperture leading into the milk flow channel and the aperture leading into the air channel are diametrically opposed relative to the axis.

Preferably, the breast shield comprises a funnel able to be positioned against a breast, the tube has a proximal end connected to the funnel and a free distal end opposite to the proximal end, a distal aperture being formed in the distal end, the distal aperture being extended by the or each aperture provided in the annular wall.

Preferably, the tube has a cylindrical internal surface extending from the proximal end to the distal end.

Preferably, the milk collection unit comprises a bayonet attachment system for attaching the tank to the connector.

Preferably, the breast shield comprises a funnel having an internal frustoconical surface extending around a first central axis and a tube extending the funnel, the tube extending around a second central axis intersecting the first central axis.

Preferably, the breast pump is suitable for being placed in a brassiere cup worn by a user. Preferably, the breast pump comprises a transit time sensor configured to detect the transit time of a signal between a transit time sensor and a level of milk contained in the tank.

Preferably, the breast pump comprises a sensor configured to measure an angle of inclination of the tank.

Also proposed is an assembly comprising the breast pump and a program configured to estimate a volume of milk contained in the tank based on the measured transit time, when the program is executed by a data processing unit.

The program can in particular be configured to estimate the volume of milk contained in the tank based on the transit time and on the angle of inclination.

### DESCRIPTION OF THE FIGURES

Other features, objects and advantages of the invention will be revealed by the description that follows, which is purely illustrative and not limiting, and which must be read with reference to the appended figures, which are the following.

FIG. 1 is a side view of a breast pump according to one embodiment.

FIG. 2 is a perspective view of the breast pump shown in FIG. 1.

FIG. 3 is a front view of the breast pump shown in FIG. 1.

FIG. 4 is a section view of the breast pump shown in FIG. 1.

FIG. 5 schematically shows electronic components of the breast pump shown in FIG. 1, as well as a terminal.

FIG. 6 is a side view of a casing according to one embodiment.

FIG. 7 is a perspective view of the casing shown in FIG. 6.

FIG. 8 is a rear view of the casing shown in FIG. 6.

FIG. 9 is a side view of a breast shield according to one embodiment.

FIG. 10 is a perspective view of the breast shield shown in FIG. 9.

FIG. 11 is a perspective view of a diaphragm according to one embodiment.

FIG. 12 is a side view of a connector according to one embodiment.

FIG. 13 is a side view of the connector shown in FIG. 12.

FIG. 14 is a section view of the connector shown in FIG. 12.

FIG. 15 is a side view of a tank according to one embodiment.

FIG. 16 is a front view of the tank shown in FIG. 15.

FIG. 17 is a top view of the tank shown in FIG. 15.

FIG. 18 is a side view of a valve according to one embodiment.

FIG. 19 is a side view of a milk collection unit according to one embodiment.

In all the figures, similar elements bear identical reference symbols.

### DETAILED DESCRIPTION OF THE INVENTION

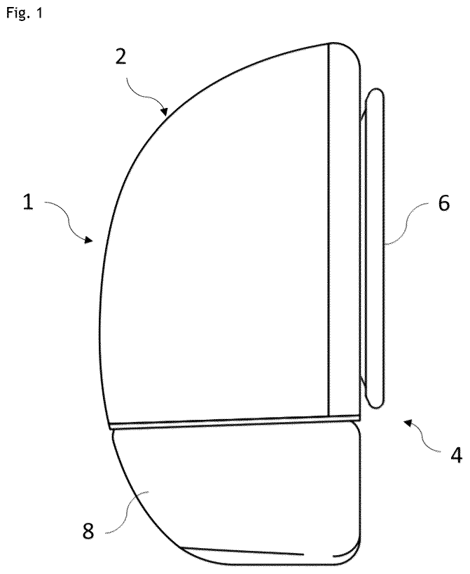

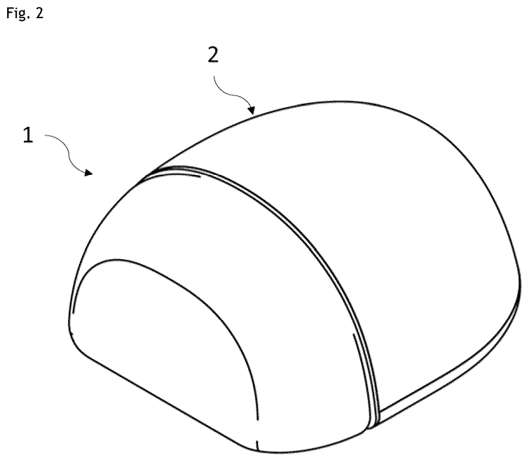

Referring to FIGS. 1 to 4, a breast pump 1 comprises a casing 2 and a milk collection unit 4. The casing 2 and the milk collection unit constitute two parts which can be attached to one another, and which can be detached from one another.

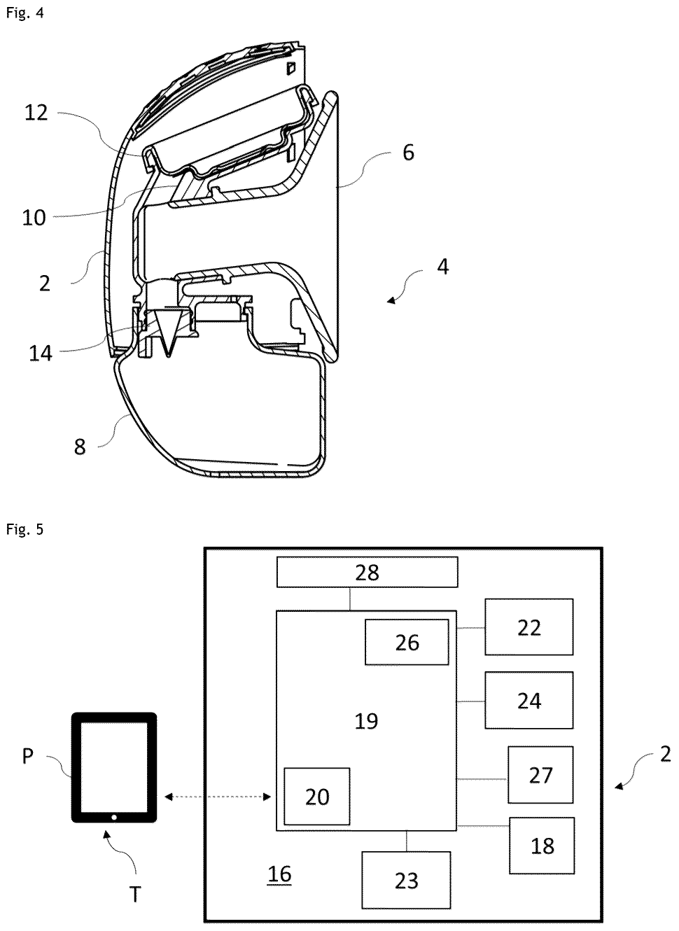

The milk collection unit 4 itself comprises several sub-parts: a breast shield 6, a tank 8, a connector 10, a diaphragm 12 and a valve 14. The connector 10, the diaphragm 12 and the valve 14 are internal components which are not visible in FIGS. 1 to 3, but which are visible in FIG. 4 showing the interior of the breast pump 1.

Referring to FIG. 5, the breast pump 1 comprises the following electronic components: a pump 18, an electronic card 19, a radio communication interface 20, a battery 22, a recharging port 23, a transit time sensor 24, an inclination sensor 26, an air valve 27 and a user interface 28. These electronic components are housed in an internal housing 16 of the casing 2. Generally, the pump 18 has the function of creating a reduced air pressure inside the breast pump 1, so as to extract milk from a breast of a user. To this end, the pump 18 can be configured to suck air in an intermittent and cyclic manner. Thus, a cycle of operation of the pump 18 comprises two phases: a suction phase during which the pump 18 sucks air thus creating a reduced air pressure in an articulation zone between the diaphragm 12 and the casing 2, delimited by a base 43 of the casing 2, and a passive phase during which the pump does not suck air.

The air valve 27 can assume two states: an open state allowing the introduction of air via the air connection 42 in the articulation zone between the diaphragm 12 and the casing 2, and a closed state not allowing this introduction.

The air valve 27 has the function of compensating the reduced air pressure caused by the pump 18 during the suction phase. To accomplish this compensation, the air valve 27 is configured in its open state during the passive phase of the pump 18 and in its closed state during the suction phase of the pump 18.

The electronic card 19 is configured to synchronize the operation of the pump 18 and of the air valve 29.

The radio communication interface 20 is suitable for establishing a wireless radio communication channel with a terminal located at a distance from the breast pump 1. The type of radio communication provided by the interface 20 is of any type, Bluetooth for example. The communication interface 20 is arranged on the electronic card 19.

The battery 22 has the function of feeding the electronic components of the breast pump 1. The battery 22 is for example rechargeable through the recharging port 23 formed on the casing 2 so as to be accessible from the outside. The recharging port 23 is for example a USB port.

The transit time sensor 24 is configured to detect the transit time of a wave propagating in the tank 8.

The inclination sensor 26 is configured to measure an angle of inclination of the tank 8. The inclination sensor is for example an inertial sensor, for example an accelerometer.

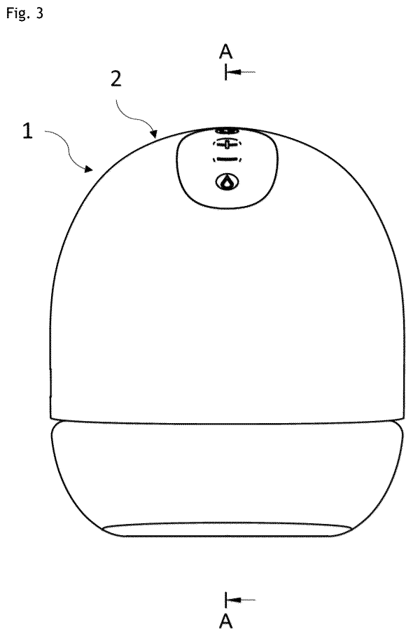

The user interface 28, also visible in FIG. 3, comprises a button allowing a user to start the pump 18 and to stop the pump 18, a button for controlling the intensity of the pumping accomplished by the pump 18, and a button for selecting between two suction programs (modes). Moreover, the user interface 28 comprises indicator lights allowing indicating to the user whether the pump 18 is started as well as the regulated intensity. The buttons and the indicator lights are arranged on the casing 2.

Hereafter, reference is made to elements of the breast pump 1 which are qualified as distal and proximal. These adjectives refer implicitly to the distance which separates these elements from a user. In other words, an element qualified as proximal is intended to be placed closer to a breast than a corresponding element qualified as distal.

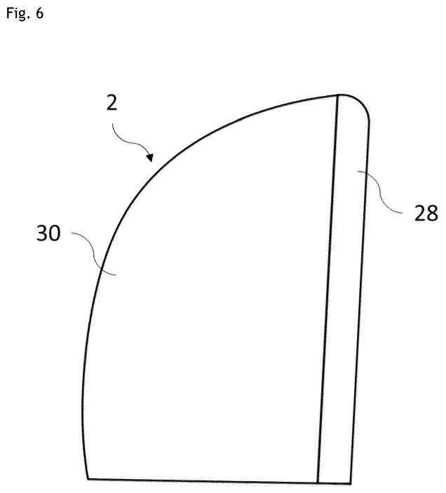

Referring to FIGS. 6 to 8, the casing 2 comprises two parts: a base 28 and a shell 30 delimiting between them the internal housing 16.

The shell 30 has an external surface and an internal surface opposite to the external surface.

The external surface of the shell 30 has a domed shape, i.e. convex in two orthogonal directions. Such a shape allows conforming to the concave internal surface of the cup of a brassiere. The internal surface of the shell 30 delimits the aforementioned internal housing 16.

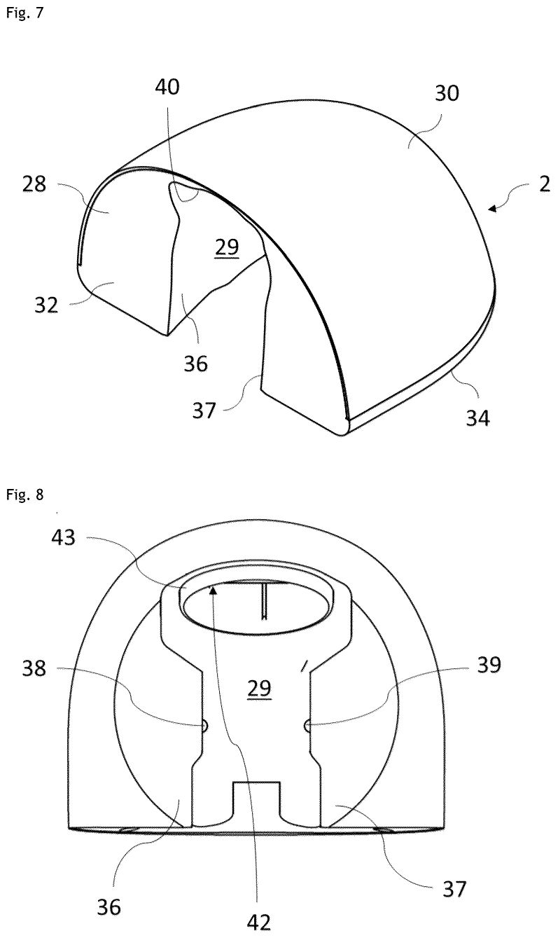

The base 28 also has an external surface and an internal surface opposite to the external surface. The external surface is concave, so as to form a trough 29 accessible from the outside of the casing 2, visible in particular in FIG. 7. The base 28 separates the internal housing 16 from the trough 29. The internal surface of the base 28 delimits the internal housing 16.

The base 28 comprises a lower wall 32. The lower wall 32 is substantially planar, and extends in a first plane which is conventionally called the horizontal plane. The lower wall 32 delimits a lower access to the trough 29. An object moved in translation in a direction normal to the first plane can thus enter into the trough 29 via the lower access.

Moreover, the base 28 comprises a proximal wall 34. The proximal wall 34 extends transversely to the lower wall 32. The proximal wall 34 extends in a second plane perpendicular to the first plane. The proximal wall 34 delimits a proximal access to the trough 29. An object moved in translation in a direction normal to the second plane can thus enter into the trough 29 via the proximal access.

It should be noted that there exists no physical separation between the proximal access and the lower access. It is thus possible to insert the milk collection unit 4 into the trough 29 in such a manner that different parts of the unit 4 cross respectively and simultaneously the proximal access and the lower access.

The base 28 also comprises two lateral walls 36, 37 delimiting between them the trough 29. The two walls 36, 37 extend parallel to a third plane perpendicular to the first plane and to the second plane. The two lateral walls 36, 37 are connected to the lower wall 32 as well as to the proximal wall 34.

The casing 2 comprises a first ball spring plunger 38. The first ball spring plunger 38 is arranged in the lateral wall 36 of the connector 10. The first ball spring plunger 38 comprises a ball mounted on a spring. The spring drives the ball toward a rest position in which the ball protrudes out of the lateral wall 36 into the trough 29. The ball can be retracted by being acted on inside the lateral wall 36, causing a compression of the spring. The ball returns naturally to its rest position once the external action ceases, through extension of the spring.

The casing 2 comprises a second ball spring plunger 39 arranged in the lateral wall 37 of the connector 10. The second ball spring plunger 39 has the same characteristics as the first ball spring plunger 38.

The first ball spring plunger 38 and the second ball spring plunger 39 are arranged so as to be facing one another in the trough 29.

The base 28 also comprises a distal wall forming the bottom of the trough 29. The distal wall connects one of the lateral walls 36 to the other lateral wall 37.

The air connection 42 is provided in the distal wall. The air connection 42 forms an aperture leading into the external surface of the base 28, hence into the trough 29, and also leading into the internal surface of the base 28, hence into the internal housing 16.

The air connection 42 is fluidly connected to the pump 18 and to the air valve 27.

Moreover, the casing 2 comprises a boss 43 protruding into the trough 29. The boss 43 is annular and extends around the air connection 42.

The different parts of the milk collection unit 4 will now be described.

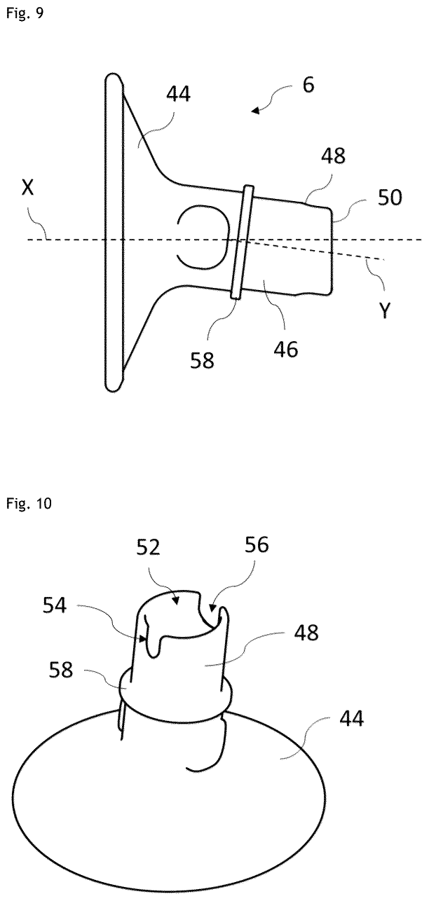

Referring to FIGS. 9 and 10, the breast shield 6 comprises a funnel 44 intended to be placed against the breast of a user, so that the nipple of the breast is placed inside the funnel 44. The funnel 44 has an internal frustoconical surface extending around a central axis X. the internal surface preferably has a flare angle greater than 100 degrees, for example on the order of 115 degrees. This allows obtaining a relatively shallow funnel 44, hence having a reduced axial bulk.

Moreover, the breast shield 6 comprises a tube 46 which extends the funnel 44.

Generally, the tube 46 delimits a tunnel into which the nipple of a breast can be partially inserted, when the breast is positioned against the internal surface of the funnel 44.

The tube 46 comprises an annular wall which extends around an axis. This annular wall comprises an internal surface with a cylindrical shape. This internal surface delimits the tunnel.

Preferably, the axis Y of the tube 46 and the axis of the funnel 44 are not conflated, but rather intersecting. In this manner, when the axis of the tube 46 is oriented horizontally, the funnel 44 can be oriented slightly downward, which is more comfortable for a user whose breast is placed in the funnel 44. Indeed, a configuration of this type allows imitating the position of a breastfed child under the median line of the nipple and of the areolar tissue of a breast.

The tube 46 has a proximal end forming a junction with the funnel 44, and a free distal end 50, opposite to the proximal end. The tube 46 has at its distal end 50 a distal aperture 52 (not visible in FIG. 9, but visible in FIG. 10). The distal aperture 52 is delimited by the internal surface of the annular wall. When this surface is axisymmetric, the distal aperture 52 is circular. The distal aperture 52 constitutes a tunnel exit. Thus milk extracted from a nipple inserted into the tunnel via the proximal end can flow into the tunnel delimited by the tube 46 in a flow direction substantially parallel to the axis of the tube 46, and can leave the tunnel and more generally the breast shield 6 by the distal aperture 52.

It should however be noted that two additional apertures 54, 56 are provided in the annular wall 48 of the tube 46.

The additional apertures extend the distal aperture 52.

The two additional apertures 54, 56 are diametrically opposite to one another relative the axis of the tube 46. They have for example an oblong shape, i.e. elongated and rounded at their ends.

The aperture 56 is a lower aperture, in that it has the purpose of being placed below the axis of the tube 46 in an operating position of the breast pump 1. By contrast, the aperture 54 is an upper aperture, in that it has the purpose of being placed above the axis of the tube 46 in the same operating position of the breast pump 1.

Moreover, the breast shield 6 comprises a stop 58 protruding from the exterior surface of the tube 46, at the proximal end of the tube 46. The abutment forms a collar extending around the annular wall 48 of the tube 46.

The breast pump 6 is made of a flexible and durable material such as silicone, so as to improve comfort in use relative to breast shields of rigid plastic.

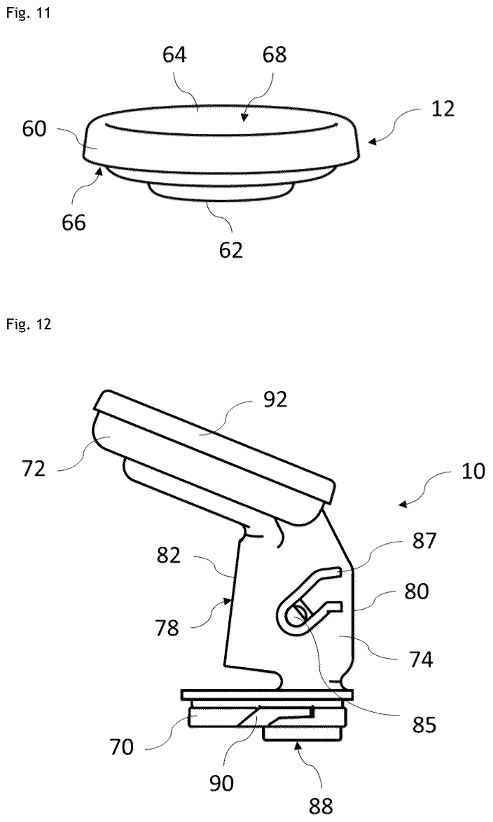

With reference to FIG. 11, the diaphragm 12 comprises a wall 60 with a circular shape. The wall 60 has a proximal surface 62 and a distal surface 64 opposite to the proximal surface 62.

The diaphragm 12 comprises an annular groove 66 formed in the proximal surface 62. For example, the annular groove is provided between two annular ribs.

The wall is made of a deformable material, such as silicone.

At rest, i.e. in a non-deformed state, the distal surface 64 is concave, so as to define a trough which can receive at least partially the boss 43 of the casing 2.

The wall 60 is suitable for passing from the resting state to a deformed state. During this deformation, the proximal surface 62 is recessed and the distal surface 64 bulges. The wall 60 is suitable for passing to the resting state by elastic return.

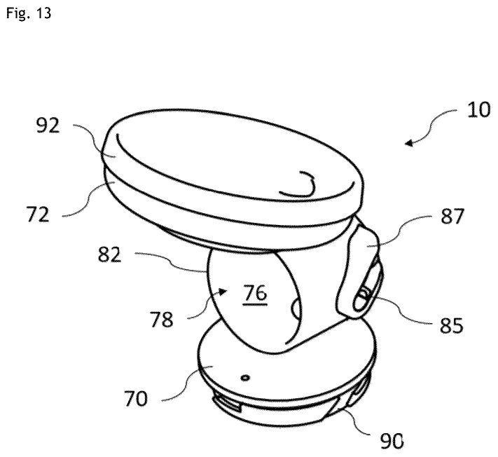

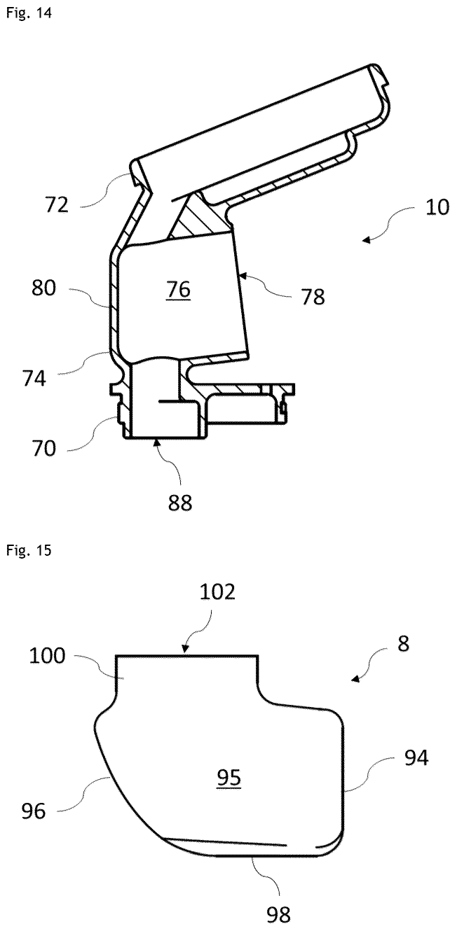

The connector 10 will now be described with reference to FIGS. 12 to 14. The connector 10 provides several functions.

First, the connector 10 serves as a support for the other parts of the milk collecting unit 4. Thus the connector 10 constitutes a part to which the breast shield 6, the tank 8 and the diaphragm 12 can simultaneously be removably attached.

Secondly, the connector 10 participates in the extraction of milk, in cooperation with the pump 18 and the diaphragm 12. We will see later how this milk extraction is carried out.

Thirdly, the connector 10 participates in conveying the milk extracted from a breast to the tank 8. To this end, the connector comprises a milk flow channel fluidly connecting the breast shield 6 to the tank 8, when the tank 8 and the breast shield 6 are attached to the connector 10.

The connector 10 comprises three parts: a lower part 70, an upper part 72 and a central part 74 arranged between the lower part 70 and the upper part 72.

The central part 74 of the connector 10 delimits a cavity 76 and a proximal access 78 to the cavity 76 from the outside of the connector 10. The cavity 76 is suitable for receiving the tube 46 of the breast shield 6 via the proximal access 78. The dimensions of the cavity 76 are suitable so that the tube 46 and the breast shield 6 are held in the cavity 76 by friction between the tube 46 and the central part 74 of the connector 10.

The central part 74 comprises a distal wall 80 forming a bottom of the cavity 76. The cavity 76 extends between the distal wall 80 and the proximal access 78.

Moreover, the central part 74 comprises a lateral wall 82 connected to the distal wall. The lateral wall 82 delimits the cavity, as well as the proximal access for the tube 46. The lateral wall 82 is annular. The lateral wall 82 has an internal surface complementary to the external surface of the tube 46, so as to create friction between these two surfaces.

Moreover, the lateral wall 82 has an external surface opposite to the internal surface.

Moreover, the connector 10 comprises a first blind hole leading into the external surface of the lateral wall 82. The first blind hole is suitable for receiving the ball of the first plunger 38 formed in the casing 2, so as to attach the connector 10 (and more generally the milk collection unit 4) to the casing 2.

Moreover, the connector 10 comprises a second blind hole 85 also leading into the external surface of the lateral wall 82. The second blind hole 85 is suitable for receiving the ball of the second plunger 39 formed in the casing 2, so as to attach the connector 10 (and more generally the milk collection unit 4) to the casing 2.

The first blind hole and the second blind hole 85 are arranged on either side of the cavity 76, symmetrically for example.

The blind holes and the ball spring plungers 38, 39 thus form means for attaching the milk collection unit 4 to the casing 2.

Moreover, the connector 10 comprises a first guide suitable for guiding the ball of the first ball spring plunger 38 toward the first blind hole. The first guide delimits a groove extending on the external surface of the lateral wall 82. The blind hole is formed at the bottom of the groove. The first guide has an entrance. The ball of the plunger can thus enter into the groove through the entrance, and be guided by the guide until it reaches the first blind hole, the first blind hole then being aligned with the ball, so that the ball penetrates into the first blind hole.

The first guide comprises an entrance part and a terminal part. The entrance part delimits the entrance. The ball is located at the bottom of the terminal part. The entrance part and the terminal part are connected by a bend. The entrance part and the terminal part are connected by a bend forming an angle comprised between 0 and 90 degrees, for example 45 degrees. Thus, when the ball of the first ball spring plunger penetrates into the groove through the entrance, it first crosses the entrance part following a substantially rectilinear trajectory, undergoes a change in direction at the bend by the aforementioned angle, then crosses the terminal part until it reaches the first blind hole.

The connector comprises a second guide 87 suitable for guiding the ball of the second ball spring plunger 39 to the second blind hole 85. The second guide 87 has the same features as the first guide.

The first guide and the second guide 87 are for example symmetrical with one another.

The lower part 70 delimits the milk flow channel, and defines in particular an exit of this channel leading to the outside of the connector 10.

The lower part 70 is configured to be removably attached to the tank 8. This removable attachment is for example provided by a bayonet attachment system 90. The bayonet attachment system 90 comprises three bent grooves provided in the lower part 70 suitable for receiving three pins of the tank 8 by a movement of translation, then of rotation so as the place the bayonet attachment system 90 in a locked position thus allowing attaching the tank 8 to the connector 10.

When the tube 46 of the breast shield 6 is inserted into the cavity of the central part 74, the lower aperture 56 provided in the annular wall of the tube 46 leads into the milk flow channel. In this position, the lower aperture 56 is even located facing the exit of the milk flow channel. In this manner, milk can flow from the lower aperture 56 of the tube 46 to the exit while flowing in a direction transverse to the axis of the tube 46.

The upper part 72 of the connector 10 serves as a support for the diaphragm 12. The upper part 72 comprises an annular rib suitable for being received in the annular groove of the diaphragm 12, so as to attach the diaphragm 12 to the connector 10 by friction.

The upper part 72 comprises an air channel. This air passage is blocked in a sealed manner by the diaphragm 12 when the diaphragm 12 is attached to the upper part 72. When the tube 46 of the breast shield 6 is inserted into the cavity formed in the central part 74 of the connector 10, the upper aperture 54 provided in the tube 46 leads into the air channel.

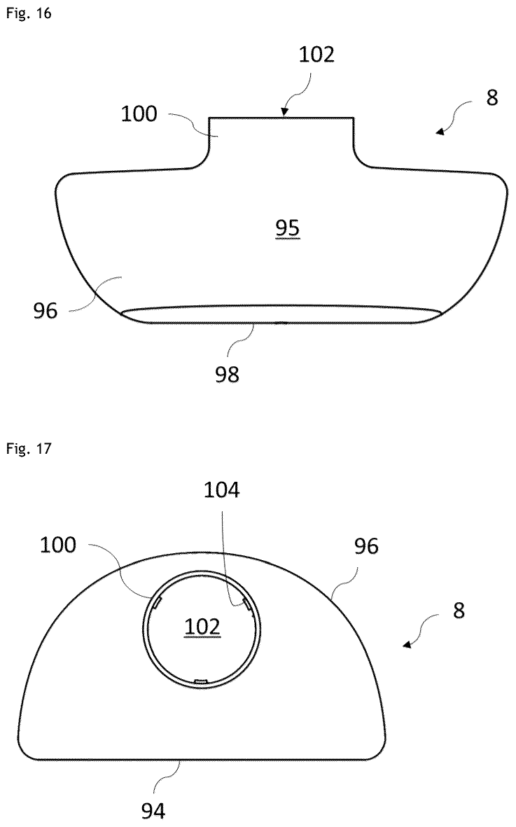

Referring to FIGS. 15 to 17, the tank 8 has the function of storing milk.

The tank comprises a proximal wall 94 and a distal wall 96, between which a milk storage space 95 is provided.

The proximal wall 94 is planar. The proximal wall 94 is arranged to extend the proximal wall of the casing 2, once the milk collection unit 4 is attached to the casing 2.

The distal wall 96 has a dome shape. The distal wall 96 is arranged to extend the shell 30 of the casing 2, once the milk collection unit 4 is attached to the casing 2.

Moreover, the tank 8 comprises a bottom wall 98 connecting the distal wall 96 to the proximal wall 94.

All or part of the walls 94, 96, 98 is translucent in order to allow observing the level of milk in the storage space 95 from the outside of the tank 8.

The tank 8 also comprises a neck 100 delimiting a milk entrance 102 through which the milk can penetrate into the milk storage space 95. The milk storage space 95 is provided between the neck 100 and the bottom wall 98.

The tank 8 comprises pins 104 suitable for cooperating with the bayonet attachment system 90 described previously, for attaching the tank 8 to the connector 10. The pins 104, three in number, protrude from the neck 100 into the entrance 102.

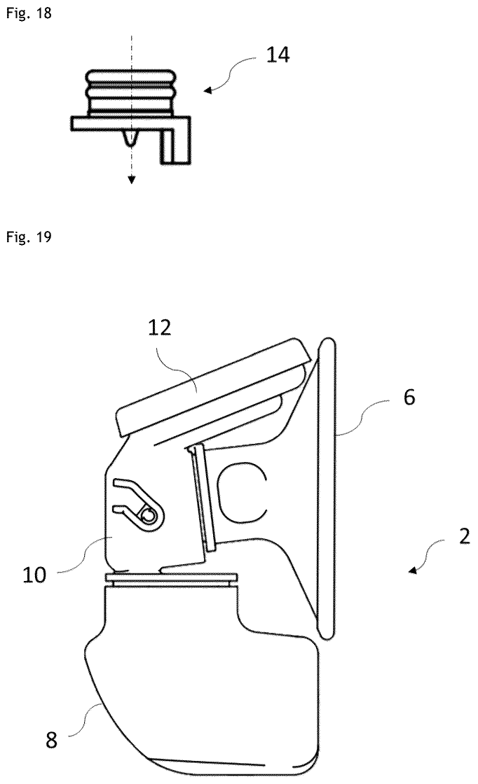

Referring to FIG. 18, the valve 14, known per se, defines a unidirectional fluid passage. Thus a fluid, milk in particular, can pass through this fluid passage in a first direction (indicated by a dotted arrow in FIG. 18), but cannot pass through this fluid passage in a second direction opposite to the first direction.

The valve 14 has dimensions suitable for being inserted into the flow channel located in the lower part 70 of the connector 10, in a direction suitable so that the unidirectional fluid passage allows milk coming from the connector 10 to penetrate into the tank 8 through the neck 100.

### Assembly of the Breast Pump

The following steps can be implemented for obtaining the breast pump 1, for example by the user of the breast pump 1, starting with the parts described previously.

In a first assembly step, the user assembles the connector 10, the breast shield 6, the tank 8, the valve 14 and the diaphragm 12, in order to form the milk collection unit 4. This first step comprises the following sub-steps.

The user attaches the breast shield 6 to the connector 10. To this end, the user inserts the tube 46 into the cavity 76 provided in the central part 74 of the connector 10, beginning with its distal end 52. The user pushes the tube 46 into the cavity 76 until the stop 58 of the breast shield 6 comes into abutment on the central part 74 of the connector 10. The breast shield 6 is then located in an end position in which the lower aperture 56 leads into the milk flow channel formed in the lower part 70 of the connector 10 and in which the upper aperture 54 leads into the air channel formed in the upper part 72 of the connector. The breast shield 6 is held in this end position by friction between the tube 46 and the lateral wall of the central part 74 of the connector 10.

Moreover, the user attaches the tank 8 to the connector 10, with the valve 14 inserted into the milk flow channel located in the lower part 70 of the connector 10. To this end, the user approaches the tank 8 to the lower part 70 of the connector 10 by a translation movement until the pins 104 formed in the neck 100 engage in the bayonet attachment system 90. Then, the user rotates the tank 8 relative to the connector 10 so as to lock the pins 104 in the bayonet attachment system 90, for example with a rotation movement of at least 45 degrees.

Moreover, the user attaches the diaphragm 12 to the connector 10. To this end, the user engages the annular rib 92 into the annular groove 66 of the diaphragm 12. The diaphragm 12 is then held on the connector 10 by friction. Once the diaphragm 12 is attached to the connector 10, the proximal surface 62 of the diaphragm 12 delimits the air channel, while the distal surface 64 is turned outward.

The milk collection unit 4 is then formed, as shown in FIG. 19. The preceding sub-steps leading to obtaining the milk collection unit 4 can be implemented in any order.

In a second assembly step, the user attaches the milk collection unit 4 to the casing 2. To this end, the user inserts the milk collection unit 4 into the trough 29 of the casing 2 by a translation movement.

During this insertion, the balls of the plungers 38, 39 formed in the lateral walls 36, 37 of the casing 2 engage in the guides 86, 87 formed on the connector 10 and are thus guided toward the blind holes, as previously indicated, so that the balls of the plungers 38, 39 engage in the blind holes, thus allowing locking the milk collection unit 4 to the casing 2. The contact of the balls with the bottom of the blind holes produces a sound which allows alerting the user to the fact that the milk collection unit 4 is indeed locked to the casing 2, which constitutes an advantage relative to other means of attachment.

During the insertion of the milk collection unit 4 into the trough 29 of the casing 2, the diaphragm 12 blocks the air connection 42 in a sealed manner. Thus the diaphragm 12 is fluidly connected to the pump 18. The diaphragm 12 is also fluidly connected to the breast shield 6, but the diaphragm 12 forms a sealed partition between the pump 18 and the breast shield 6. It should also be noted that the boss 43 of the casing 2 engages into the trough formed in the distal surface 64 of the diaphragm 12, which contributes toward stabilizing the milk collection unit 4 relative to the casing 2 and to ensuring the seal between the diaphragm 12 and the casing 2 to provide the transfer of suction to the breast shield 6.

Once the milk collection unit 4 is attached to the casing 2, the breast pump 1 is obtained and is ready to be used.

The breast pump 1 can advantageously be combined with a program P for remote control of the breast pump 1.

The program P is able to be installed in a terminal equipped with a processor for executing the program P, and with a radio communication interface 20 suitable for communicating with the radio communication interface 20 contained in the casing 2 of the breast pump 1. The program is for example an application for a smartphone or a tablet.

### Operation of the Breast Pump

The user positions the breast pump 1 in an operating position.

In the operating position, a breast of the user is positioned at least partially in the funnel 44 of the breast shield 6, so that the nipple of the breast is aligned with the tube 46. If the user is wearing a brassiere, the user can in particular place the breast pump inside the cup of the brassiere, so that the shell 30 is in contact with the cup.

In the operating position, the tank 8 is located below the casing 2 and below the connector 10. The lower part 70 of the connector 10 is located below the central part 74, and the upper part 72 is located above the central part 74.

The user then starts the pump 18. One method of starting the pump 18 consists of pressing on the corresponding button of the user interface 28 arranged on the casing 2. Another method of starting the pump 18 consists of using the terminal and the remote control program as previously described. The user interacts with the terminal in such a manner that the program generates a command for starting the pump 18. This command is transmitted by the radio communication interface 20 of the terminal, then received by the radio communication interface 20 contained in the casing 2, then relayed to the pump 18.

In operation, the pump 18 causes a deformation of the diaphragm 12. Under the influence of the pump 18 and of the air valve 29, the diaphragm 12 deforms, alternately passing into its resting state and the deformed state as described previously.

During the aspiration phase of the pump 18, the pump 18 sucks air which causes a reduced air pressure in the articulation zone between the diaphragm 12 and the casing 2, delimited by the annular boss 43 of the casing 2. Due to this reduced air pressure, the diaphragm passes from its resting state to the deformed state, i.e. the proximal surface 62 is recessed and the distal surface 64 bulges. This deformation of the diaphragm 12 causes another reduced air pressure in the air channel formed in the connector 10 and in the tube 46 of the breast shield 6. This reduced air pressure is not compensated by the valve 14, because the valve 14 prevents the arrival of fluid and particularly air inside the connector 10. The reduced air pressure thus causes translation of the nipple in the tunnel 46, this translation stimulating the physiological mechanism of ejection of the milk via the nipple located in the breast shield 6. The nipple can, during this process, undergo elongation in the tube 46 by suction. The cylindrical shape of the internal surface of the tube 46 has the advantage of avoiding injuring the nipple during this suction.

During the passive phase of the pump, the air valve 27 opens, thus allowing air to enter into the articulation zone between the diaphragm 12 and the casing 2, delimited by the annular boss 43 of the casing 2 through the air connection 42, which compensates the reduced air pressure previously created. Consequently, the diaphragm 12 returns to its resting position, the reduced air pressure in the air channel ceases, thus allowing the nipple to return to its original shape.

Milk extracted from the breast flows in the tube 46 until it reaches the lower aperture 56 provided in the annular wall of the tube 46. The milk escapes from the tube 46 through the lower aperture 56, and falls into the milk flow channel of the connector 10. The milk can then continue to fall by gravity into the channel until it reaches the exit 88 formed in the lower part 70 of the connector 10. The milk then passes through the unidirectional passage defined by the valve 14, then penetrates into the tank 8.

### Estimation of the Milk Level in the Tank

One method for estimating the level of milk stored in the storage space 95 comprises the following steps.

A transit time sensor 24 transmits a signal that propagates in the tank 8, then is reflected on the surface of the milk contained in the tank (or if milk is lacking, on the bottom wall 98). The echo resulting from this reflection is sensed by the sensor 24. The transit time sensor 24 detects the transit time of the signal.

Based on the principle that the geometry of the storage space 95 is known, and assuming that the surface of the milk is parallel to the bottom of the tank 8, it is possible to estimate the volume of milk contained in the tank 8 by a calculation using the transit time as input.

However, it should be noted that the tank 8 can be inclined in such a manner that the surface of the milk inside the tank 8 is not perfectly parallel to the bottom of the tank 8. Under these conditions, the estimate of the volume of milk on the sole basis of the transit time acquired by the transit time sensor 24 can be inaccurate.

To correct this inaccuracy, the inclination sensor 26 can be additionally used to measure an angle of inclination of the tank 8. The angle of inclination can then be combined with the transit time acquired by the transit time sensor 24 to calculate a more accurate estimate of the volume of milk.

The estimate of the volume of milk based on the measured transit time, and if applicable on the basis of the angle of inclination, can be implemented by a data processing unit integrated with the breast pump 1, contained for example in the casing 2. As a variant, this estimation step is implemented by the program installed in the terminal, after transmission of the measurements accomplished by the sensors 24, 26 to the terminal via the communication interface 20 contained in the casing 2.

### Cleaning of the Breast Pump

After use, the user can disassemble the breast pump in order to clean its different parts. The disassembly of the breast pump comprises two steps.

In a first disassembly step, the user separates the milk collection unit 4 from the casing 2. During this separation, the different parts of the milk collection unit remain assembled to one another. In particular, the breast shield and the tank remain fluidly connected by the connector 10. Consequently, no drop of milk can fall into the trough 29 of the casing 2. As the casing 2 of the breast pump is not soiled by milk, this part does not need to be cleaned as often as the milk collection unit 4.

In a second disassembly step, the user disassembles the milk collection unit 4. To this end, the user separates the breast shield 6, the tank 10, the diaphragm 12 and the valve 14 from the connector 10. It is only at this stage of this second disassembly step that a separation of the fluid connection between breast shield 6 and the tank 8 occurs. Drops of milk can of course escape from the connector during this second step, but these drops of milk cannot in principle reach the casing 2.

Once the parts of the milk collection unit are separated, the user can clean them individually before reassembling the breast pump 1 as indicated previously.

1. A breast pump comprising:
a casing,
a milk collection unit comprising a breast shield and a tank configured to be fluidly connected together, the milk collection unit being configured to:
be attached as a whole to the casing, after the breast shield and the tank have been fluidly connected together,
be separated from the casing while the breast shield and the tank remain fluidly connected together,
a pump arranged in the casing, the pump being configured to cause an extraction of milk from a breast positioned against the breast shield when the milk collection unit is attached to the casing, thereby causing a flow of milk from the breast shield to the tank.

2. The breast pump as claim in claim 1, comprising at least one ball spring plunger to attach the milk collection unit to the casing.

3. The breast as claimed in claim 1, wherein the milk collection unit comprises a connector configured to be removably attached to the tank and to the breast shield, the connector comprising a milk flow channel fluidly connecting the breast shield to the tank, when the tank and the breast shield are attached to the connector.

4. The breast pump as claimed in claim 1, wherein the milk collection unit is configured to be attached as a whole to the casing solely by the connector .

5. The breast pump as claimed in claim 3, wherein the breast shield comprises a tube inserted into the connector when the breast shield is attached to the connector, the tube comprising:
an annular wall extending around an axis,
an aperture provided in the annular wall and leading into the milk flow channel when the tube is inserted into the connector.

6. The breast pump as claimed in claim 3, wherein the milk collection unit further comprises a diaphragm configured to be deformed by the pump when the milk collection unit is attached to the casing, the deformation of the diaphragm causing a reduced air pressure in the air channel when a breast is positioned against the breast shield.

7. The breast pump as claimed in claim 6, wherein the diaphragm comprises an annular groove, and the connector comprises an annular rib able to be engaged in the annular groove so as to attach the diaphragm to the connector.

8. The breast pump as claimed in claim 6, wherein the connector comprises an air channel fluidly connecting the breast shield to the diaphragm, when the breast shield is attached to the connector.

9. The breast pump as claimed in claim 8, wherein the breast shield comprises a tube inserted into the connector when the breast shield is attached to the connector, the tube comprising:
an annular wall extending around an axis,
an aperture provided in the annular wall and leading into the air channel when the tube is inserted into the connector.

10. The breast pump as claimed in claim 9, wherein
the tube comprises a further aperture provided in the annular wall and leading into the milk flow channel when the tube is inserted into the connector
the further aperture leading into the milk flow channel and the aperture leading into the air channel are diametrically opposed relative to the axis.

11. The breast pump as claimed in claim 5, wherein the breast shield comprises a funnel able to be positioned against a breast, the tube has a proximal end connected to the funnel and a free distal end opposite to the proximal end, a distal aperture being formed at the distal end, the distal aperture being extended by the or each aperture provided in the annular wall.

12. The breast pump as claimed in claim 11, wherein the tube has a cylindrical internal surface extending from the proximal end to the distal end.

13. The breast pump as claimed in claim 12, wherein the milk collection unit comprises a bayonet attachment system for attaching the tank to the connector.

14. The breast pump as claimed in claim 11, wherein the breast shield comprises:
a funnel having an internal frustoconical surface extending around a first central axis,
tube extending the funnel, the tube extending around a second central axis intersecting the first central axis.

15. The breast pump as claimed in claim 1, configured to be placed in a brassiere cup worn by a user.

16. The breast pump as claimed in claim 1, wherein the casing comprises a base and a shell delimiting between them an internal housing wherein the pump is arranged.

17. The breast pump as claimed in claim 16, wherein the base has an internal surface delimiting an internal housing and an external surface opposite to the internal surface, the external surface delimiting a cavity, the milk collection unit being partially received in the cavity when the milk collection unit is attached to the casing.

18. The breast pump as claimed in claim 1, comprising a transit time sensor configured to detect the transit time of a signal between the transit time sensor and a milk level contained in the tank.

19. An assembly comprising:
a breast pump as claimed in claim 1,
a computer program configured to estimate a volume of milk contained in the tank based on the measured transit time, when the program is executed by a data processing unit.

20. The assembly as claimed in claim 19, comprising a sensor configured to measure an angle of inclination of the tank, and wherein the computer program is configured to estimate the volume of milk contained in the reservoir based on the transit time and on the angle of inclination.
